(19) United States
(12) Patent Application Publication
(10) Pub. No.: US 2003/0065027 A1
(43) Pub. Date: Apr. 3, 2003

(54) Compositions for controlling microorganisms, comprising primary and secondary esters of polyglycerol in an effective ratio

(76) Inventors: Achim Brock, Essen (DE); Burghard Gruning, Essen (DE); Geoffrey Hills, Essen (DE)

Correspondence Address: FROMMER LAWRENCE & HAUG LLP, 745 Fifth Avenue, New York, NY, 10151, US

(21) Appl. No.: 10/126,186

(22) Filed: Apr. 18, 2002

(30) Foreign Application Priority Data
Apr. 20, 2001 (DE) 101 19 682.2

(51) Int. Cl. A61K 31/225

(52) U.S. Cl. 514/547; 514/552

(57) ABSTRACT

The invention relates to compositions for controlling microoganisms, comprising primary and secondary esters of polyglycerol in an effective ratio of from 8:1 to 25:1.

### RELATED APPLICATIONS

This application claims priority to German Application 101 19 682.2, filed Apr. 20, 2001, incorporate herein by reference.

### BACKGROUND OF THE INVENTION

1. Field of the Invention

The invention relates to compositions for controlling microorganisms, comprising primary and secondary esters of polyglycerol in an effective ratio.

2. Background of the Invention

A large number of antimicrobial chemical substances and mixtures of these substances are known for controlling microorganisms (Gram-positive bacteria, Gram-negative bacteria, mycobacteria, dermatophytes, yeasts, fungi and hyphal fungi, viruses and spores) which are present on the surface of skin and hair, clothing, devices for body cleansing and bodycare such as, for example, in the dental sector, medical instruments, but also rooms and fitments; said substances and mixtures are divided according to their intended use into disinfectants, preservatives, antiseptics and cosmetic active ingredients, to name but a few.

The main representatives of these groups are: aldehydes, such as formaldehyde, glyoxal or glutaraldehyde; phenol derivatives, such as 2,2′-dihydroxybiphenyl and 4-chloro-3-methylphenol; quaternary ammonium compounds, cationic surfactants, such as benzalkonium chloride, cetrimonium bromide, cetylpyridinium chloride; amphoteric surfactants, and also compounds which release active oxygen, such as, for example, hydrogen peroxide, organic peracids, alkyl peroxides or alkyl hydroperoxides.

However, these compounds have a number of disadvantages since they do not meet, or only meet inadequately, the diverse requirements which are placed on them in practice, such as, for example broad activity spectrum, short contact times at low temperatures, good skin compatibility, low toxicity, material compatibility.

Aldehyde- or phenol-based disinfectants are regarded as being toxicologically and ecologically unacceptable, often lead to sensitizations, in particular of the skin and respiratory organs, and moreover have a characteristic, pungent and unpleasant odor. Some are also potential carcinogens.

Quaternary ammonium compounds (quats) are for the most part toxicologically acceptable, have no or only very low skin sensitization and are virtually odorless. However, they have a considerable skin-irritative effect. As in the case of the use of aldehydes, the use of quats may lead to undesired deposits and films on the surfaces treated; these are optically disadvantageous and can only be removed again by customary cleansing processes with difficulty or not at all.

DE-A-42 37 081 discloses cosmetic deodorants which comprise, as active ingredients, fatty acid esters of di- and triglycerol prepared by chemical means. According to the teaching therein, only the primary monoesters of diglycerol (substitution in the 1-position) and the secondary monoesters of triglycerol (selective substitution in the 2′-position) are effective for controlling Gram-positive bacteria.

These regioisomerically pure monoesters can be prepared according to known chemical processes of the prior art (DE-A-38 18 293) by alkaline-catalyzed reaction of a 1.5 to 2.5-fold molar excess of fatty acids or fatty acid derivatives with isopropylidene derivatives of di- and triglycerol, subsequent purification of the reaction product and subsequent acidic hydrolysis or alcoholysis of the isopropylidene groups. When the reaction is complete, the solution has to be neutralized and the monoesters have to be isolated and purified. As well as the multistage nature of the synthesis, based on the need for protective group chemistry, in the case of diglycerol derivatives, the use of equimolar amounts of epichlorohydrin is additionally to be regarded as a disadvantage of this route.

In addition, enzymatically catalyzed processes for the preparation of primarily substituted polyglycerol fatty acid esters are also known. In this connection, D. Charlemagne and M. D. Legoy (JAOCS 1995, Vol. 72, No. 1, 61-65) adsorb firstly the polyglycerol to the same amount of silica gel before allowing it to react in suspension with fatty acid methyl esters with lipase catalysis. The main disadvantage here is the loss of the expensive enzyme which is separated off together with the silica gel by filtration when the reaction is complete. In addition, they reported, in agreement with the prior art (R. Lortie, M. Trani, F. Ergan, Biotechnol. Bioeng. 1993, 41, 1021), that when 1,3-specific lipases are used, isomerizations by acyl migration from primary to secondary positions are observed. S. Matsumura, M. Maki, K. Toshima and K. Kawada (J. Jpn. Oil Chem. Soc. 1999, Vol. 48, No. 7, 681-692) utilize a modification of this process in order to synthesize polyglycerol esters using 20% by weight of enzyme. According to the teaching given in DE-A-42 37 081, they carry out further purification at high expenditure by means of column chromatography in order to obtain pure monoesters with the known antimicrobial activities.

Thus, in summary, the teachings of the prior art imply that only the primary diglycerol monoesters and the secondary triglycerol monoesters are suitable agents for controlling microorganisms. These compounds are obtainable with high preparative complexity. Mixtures of regioisomers of pure monoesters are known. They have comparable antimicrobial properties. However, their preparation is likewise associated with high synthesis and purification complexity.

### OBJECTS OF THE INVENTION

It is therefore an object of the invention to find compositions for controlling microorganisms which largely remedy the described disadvantages of the compositions of the prior art, display high antimicrobial action and can be prepared in an uncomplicated manner from readily accessible raw materials by an economically feasible and ecologically acceptable process.

It was surprising and could not have been foreseen by the person skilled in the art on the basis of the teachings of the prior art that mixtures of fatty acid mono-, di- and triesters of polyglycerol which in each case comprise primary and secondary mono-, di- and triesters of polyglycerol in an effective ratio also have comparable and sometimes even significantly better activities in the control of microorganisms than the monoesters prepared by chemical synthesis or enzymatic preparation and purification.

### SUMMARY OF THE INVENTION

The invention therefore provides compositions for controlling microorganisms which have a content of mixtures of fatty acid esters of polyglycerol comprising primary and secondary esters of polyglycerol in an effective molar ratio, and which can be prepared simply in one step from the raw materials polyglycerol and fatty acid or fatty acid derivative.

The invention further provides for the use of antimicrobial mixtures of fatty acid monoesters and fatty acid diesters of polyglycerol which have a content of primary and secondary esters of polyglycerol in an effective ratio, in particular of di- and/or triglycerol, for the preparation of disinfectants, sterilizing compositions, antiseptics, preservatives which are suitable for the sterilization and disinfection of surfaces and surgical instruments, and preservation, in particular for preservation of cosmetic or dermatological compositions. Antimicrobial substances included, for example Gram-positive bacteria, Gram-negative and hyphal fungi, viruses and spores.

Moreover, the compositions are also suitable for the preservation of foods and can also be used for the antimicrobial finishing of food packagings.

The antimicrobial compositions according to the invention are particularly suitable, due to their mildness, for the preparation of cosmetic preparations for controlling body odor, for controlling dandruff and for controlling blemished skin.

### BRIEF DESCRIPTION OF THE FIGURES

FIG. 1 is the gas chromatograph for diglycerol caprate from Example 1.

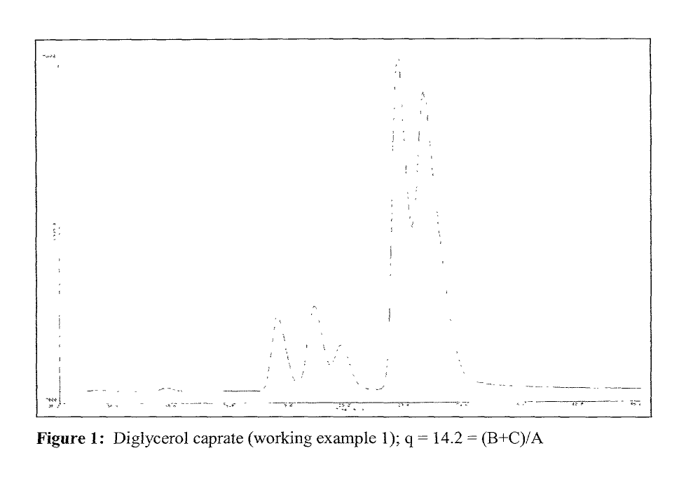

FIG. 2 is the gas chromatograph of D-Caprate A obtained from Solvay Alkali GmbH.

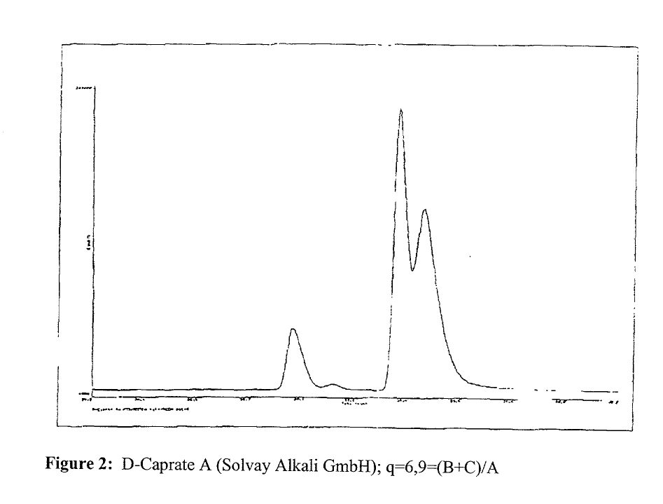

FIG. 3 is the gas chromatograph of diglycerol caprylate from Example 3.

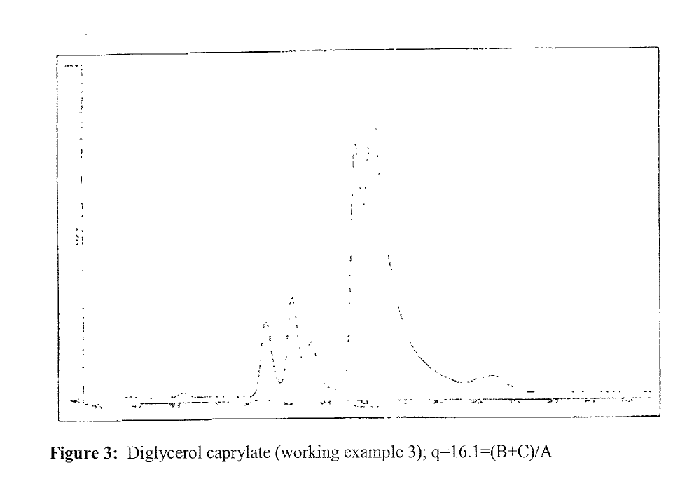

FIG. 4 is the gas chromatograph of polyglycerol-3-caprylate from Example 4.

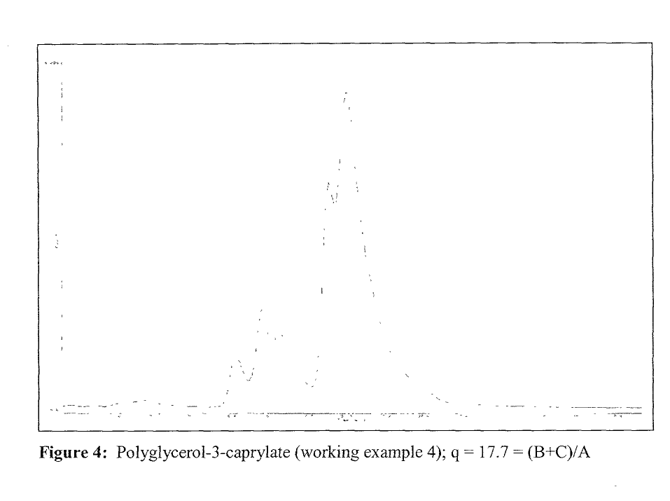

FIG. 5 is a graph depicting the effectiveness of diglycerol monocaprate not according to the invention against Corynebacterium xerosis, Staphylococcus epidermidis, and Candida albicans.

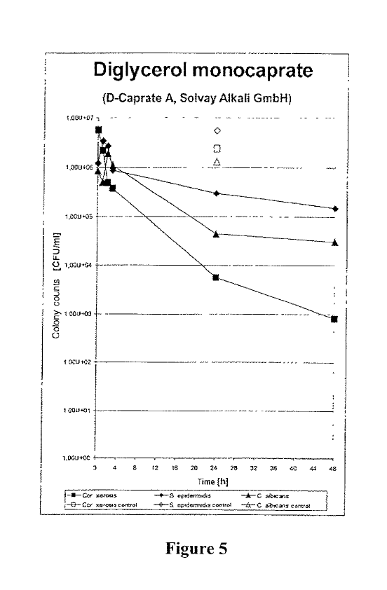

FIG. 6 is a graph depicting the effectiveness of diglycerol caprate from Example 1 depicting the effectiveness of diglycerol monocaprate not according to the invention against Corynebacterium xerosis, Staphylococcus epidermidis, and Candida albicans.

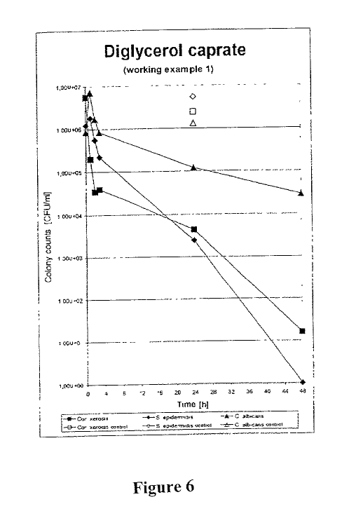

FIG. 7 is a graph depicting the effectiveness of polyglycerol-3-caprate from Example 2 depicting the effectiveness of diglycerol monocaprate not according to the invention against Corynebacterium xerosis, Staphylococcus epidermidis, and Candida albicans.

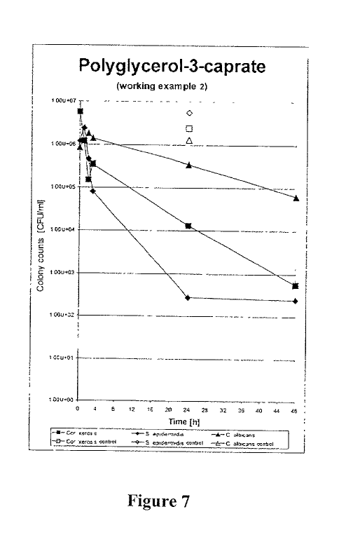

FIG. 8 is graph depicting the effectiveness of diglycerol caprylate from Example 3 depicting the effectiveness of diglycerol monocaprate not according to the invention against Cornyebacterium xerosis, Staphylococcus epidermidis, and Candida albicans.

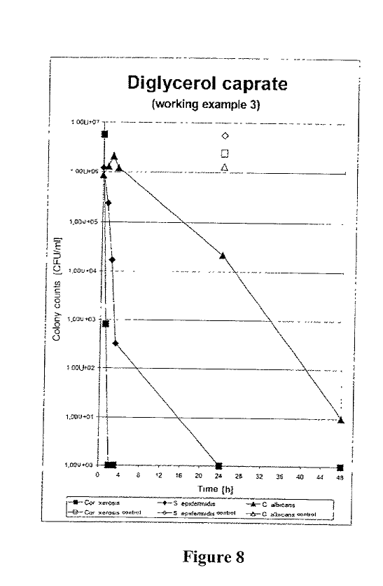

FIG. 9 is a graph depicting the effectiveness of polyglycerol-3-caprylate from Example 4 depicting the effectiveness of diglycerol monocaprate not according to the invention against Corynebacterium xerosis, Staphylococcus epidermidis, and Candida albicans.

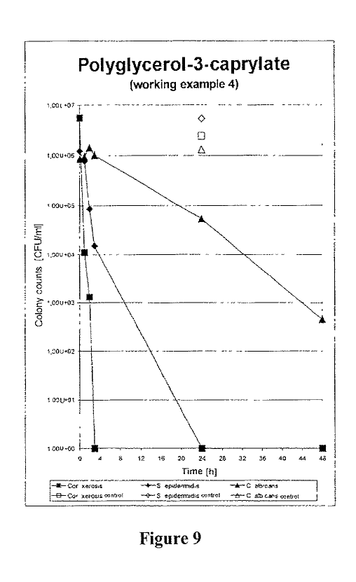

FIG. 10 is a graph depicting the effectiveness of triglycerol monolaurate obtained commercially depicting the effectiveness of diglycerol monocaprate not according to the invention against Corynebacterium xerosis, Staphylococcus epidermidis, and Candida albicans.

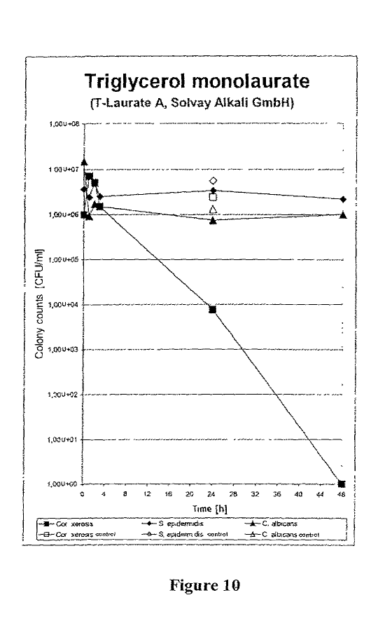

FIG. 11 is a graph depicting the effectiveness of triglycerol laurate from Example 5 depicting the effectiveness of diglycerol monocaprate not according to the invention against Corynebacterium xerosis, Staphylococcus epidermidis, and Candida albicans.

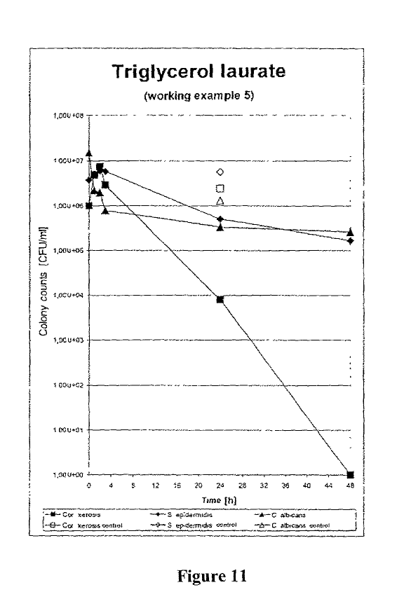

### DETAILED DESCRIPTION

The polyglycerols used according to the invention are, firstly, linear compounds of the general formula

HO—CH₂—CH(OH)—CH₂—O—[CH₂—CH(OH)—CH₂—O]ₙ—H

in which n=1-9, preferably 1-6, in particular 1-3, specifically 1 and 2. Moreover, the polyglycerols used can also be branched and contain cyclic proportions. They are liquids which are highly viscous at room temperature and which, in addition to diglycerol, primarily comprise the more highly condensed oligomers of glycerol. For the purposes of the present invention, particular preference is given to using technical-grade mixtures of polyglycerols which usually comprise diglycerol, triglycerol, tetraglycerol and pentaglycerol.

They can, for example, be prepared industrially by base-catalytic condensation of glycerol or else by hydrolysis and condensation of epichlorohydrin. Moreover, polyglycerols are also accessible by polymerization of glycidol. Separation and isolation of the individual polyglycerols is possible by treatment with the various means known in the prior art. An overview by G. Jakobson of the various synthetic routes can be found in “Fette Seifen Anstrichmittel”, 1986, volume 88, No. 3, 101-106. The various structural possibilities for polyglycerol can be checked in H. Dolhaine, W. Preuβ and K. Wollmann (Fette Seifen Anstrichmittel 1984, volume 86, No. 9, 339-343).

Commercially available products are generally mixtures of polyglycerols with varying degrees of condensation; their maximum degree of condensation can usually be up to 10 and in exceptional cases may be even greater. They comprise about 0 to 5% by weight of glycerol, 15 to 40% by weight of diglycerol, 30 to 55% by weight of triglycerol, 10 to 25% by weight of tetraglycerol, 0 to 10% by weight of higher oligomers.

The polyglycerols preferably used according to the invention comprise about 15 to about 35% by weight of diglycerol, about 38 to about 52% by weight of triglycerol, about 15 to about 25% by weight of tetraglycerol, <about 10% by weight of higher oligomers and <about 2% by weight of cyclic compounds. Particular preference is given to using polyglycerols which comprise only or predominantly diglycerol.

The fatty acids and fatty acid derivatives, and mixtures thereof, to be used with preference for the purposes of the present invention are derived from straight-chain or branched, saturated, mono- or polyunsaturated carboxylic acid and fatty acid radicals preferably having 6 to 14 carbon atoms, more preferably 8 to 12, in particular 8 to 10, carbon atoms in the main chain and, optionally, contain OH groups and/or double bonds.

The fatty acid derivatives which may be used are all customary derivatives which take part in (trans)esterification reactions. According to the invention, the fatty acid derivatives are particularly preferably chosen from fatty acid alkyl esters having 1 to 4 carbon atoms in the alcohol radical.

The fatty acids or esters thereof used are, individually or in mixtures, fatty acids, such as caproic acid, caprylic acid, capric acid, 2-ethylhexanoic acid, undecylenic acid, lauric acid and myristic acid. In principle, all fatty acids with a similar chain distribution are suitable.

Preference is given to using caprylic acid and capric acid.

The quantitative ratio of fatty acid or fatty acid derivatives to polyglycerol is set so that there is an excess of hydroxyl groups compared with fatty acid radicals in the reaction mixture. For the purposes of the present invention, preference is given to setting the quantitative ratio of moles of fatty acid derivatives to moles of polyglycerol to a ratio from about 0.25:1 to about 4:1, in particular about 0.5:1 to about 2:1.

The (trans)esterification reaction to give the antimicrobial mixtures of fatty acid monoesters and fatty acid diesters of polyglycerol which have a content of primary and secondary esters of polyglycerol in an effective ratio can be carried out by means of enzymes, in particular immobilized enzymes, preferably with those enzymes chosen from the group of lipases, esterases or proteases, in particular lipases. They have enzyme catalysis activity for ester bonds, in particular for hydrolysis, esterification and transesterification. Such lipases are described in WO 90/09451. Moreover, the product Novozym® 435 from Novozymes as an immobilized lipase system is known and commercially available. This enzyme is particularly preferably used for the purposes of the present invention.

A gas chromatographic and a nuclear magnetic resonance spectroscopic method for the analysis and characterization of partially esterified polyglycerols is described by R. Gerhards (paper “Trends in der Analytik von kosmetischen Rohstoffen” [Trends in the analysis of cosmetic raw materials] at the 14th DGK symposium “Innovative Analytik in der Kosmetik—Anforderungen, Applikationen, Trends” [Innovative analysis in cosmetics—requirements, applications, trends], 04.-06.2001, Congress Centrum Hamburg). The combination of the methods presented therein is suitable for identifying and quantifying the primary and secondary monoesters of di- and polyglycerol and is thus particularly suitable for characterizing the effective ratio of primary and secondary isomers of the polyglycerol esters according to the invention. In the case of diglycerol monoesters in particular, gas chromatography gives a precise quantitative statement concerning the ratio of isomeric primary to secondary esters of linear diglycerol. In this connection, the diglycerol monoesters can be present in pure form, in mixtures of diglycerol esters of varying degree of esterification, and also as a constituent of a mixture of different polyglycerols and different degrees of esterification. Forming the quotient of the areal percentages of defined peaks in the retention range for the diglycerol monoester reveals the molar ratio of primary to secondary monoester isomers of linear diglycerol.

In summary, the polyglycerol fatty acid esters according to the invention consist of a mixture of compounds of varying degree of esterification, each degree of esterification being composed of a mixture of primarily and secondarily substituted isomers in an effective ratio. Considerable proportions of nonesterified polyglycerol may be present. The polyglycerol which is used as a basis can here be uniform or for its part again a mixture of products of varying degree of condensation.

Moreover, the compositions according to the invention for controlling microorganisms can, depending on the intended use, also comprise anionic, nonionic, cationic and/or amphoteric surfactants customary in this field.

Typical examples of such surfactants are:

1. nonionic surfactants based on alkylene oxides, such as ethoxylates of long-chain branched alcohols, ethoxylates of sorbitan esters, propylene oxide-ethylene oxide copolymers, hydroxyalkyl fatty acid amides, polydimethylsiloxane-polyalkylene oxide copolymers, sugar-based surfactants, such as alkyl polyglycosides, alkyl glycoside esters, N-acylglucamides and polyglycerol esters,

2. anionic surfactants, such as alkyl sulfates and alkyl ether sulfates, α-olefinsulfonates, fatty acid ester sulfonates, alkylarylsulfonates, sulfosuccinates, alkyl or alkoxyalkyl phosphates, taurates, N-acylamino acid derivatives, sarcosinates, isethionates and soaps,

3. cationic surfactants, such as alkyltrimethylammonium salts, fatty acid esters of di- or triethanolammonium salts, alkylimidazolinium salts, acylamidopropyl-dimethylammonium salts, cationically derivatized polydimethylsiloxanes,

4. zwitterionic and amphoteric surfactants, such as betaines, sulfobetaines, amine oxides and amphoacetates.

The compositions according to the invention for controlling microorganisms are, for example, sterilizing compositions, disinfectants, disinfectant cleaning compositions, all-purpose cleaners, sanitary cleaners, bath cleaners, machine dishwashing detergents, laundry detergents, cosmetic cleansers and care compositions. Cosmetic compositions based on the described polyglycerol fatty acid esters are used, in particular, for controlling body odor, dandruff or for controlling skin blemishes. They can be formulated as such in the form of homogeneous liquids, as gels, as ointments, as wax-like or emulsion-like preparations. Particularly in the emulsion form, they comprise oils, such as ester oils, volatile or low-volatile silicone derivatives, such as decamethylcyclopentasiloxane, paraffin oils and the like.

It may be advantageous to co-use other antimicrobial substances in the compositions according to the invention for controlling microorganisms. As such, mention may be made of triclosan, farnesol, glycerol monolaurate or 2-ethylhexyloxyglycerol. Depending on the intended use, as well as said surfactants, they may also comprise the auxiliaries and additives specific in each case, for example solvents, builders, foam inhibitors, salts, bleaches, bleach activators, optical brighteners, graying inhibitors, solubilizers, thickeners, fragrances and dyes, emulsifiers, biogenic active ingredients, such as plant extracts and vitamin complexes. Suitable solvents are, in particular, water or alcohols, such as, for example, ethanol, propanol, isopropanol, 2-methyl-2-propanol, propylene glycol, dipropylene glycol or glycerol.

The amounts of such additives to be used in each case are, depending on the nature of the respective product, known to the person skilled in the art or, where necessary, can be readily determined by simple experimentation.

Other possible uses for the compositions according to the invention is their use as preservatives in foods and in food packagings, where they are usually used in concentrations of from about 0.01 to about 5% by weight, preferably about 0.1 to about 1% by weight. The esters according to the invention can simply be added to foods in the corresponding amount. The polyglycerol esters are used in packaging by, for example, impregnating papers with a solution or emulsion of the esters, or by spraying films with corresponding preparations of the esters. The esters can also be added before or during the shaping process of the packagings, such as extrusion.

### EXAMPLES

The working examples below represent preferred reactions of the present invention, but are not suitable for limiting the invention thereto.

### Example 1

Diglycerol caprate

415 g of diglycerol (obtainable from Solvay Alkali GmbH) and 431 g of capric acid were weighed into a three-necked flask fitted with precision glass stirrer and attached distillation bridge, and 16.9 g of Novozym® 435 were added at 60° C. The water of reaction which formed was removed in a water-jet vacuum until the acid number of the reaction mixture dropped to a value below 2. To separate off the enzyme, the product was finally filtered.

### Example 2

Polyglycerol-3 caprate

460 g of a polyglycerol characterized by the following distribution (% by weight): 0.2 of glycerol, 32.6 of diglycerol, 41.2 of triglycerol, 14.8 of tetraglycerol, 3.9 of pentaglycerol, 1.9 of hexaglycerol, 5.4 of higher polyglycerols and 345 g of capric acid are weighed into a three-necked flask fitted with precision glass stirrer and attached distillation bridge, and 16.1 g of Novozym® 435 were added at 60° C. The water of reaction which forms is removed in a water-jet vacuum until the acid number of the reaction mixture has dropped to a value below 2. To separate off the enzyme, the product was finally filtered.

### Example 3

Diglycerol caprylate

415 g of diglycerol (obtainable from Solvay Alkali GmbH) and 361 g of caprylic acid were weighed into a three-necked flask fitted with precision glass stirrer and attached distillation bridge, and 15.5 g of Novozym® 435 were added at 60° C. The water of reaction which forms was removed in a water-jet vacuum until the acid number of the reaction mixture has dropped to a value below 2. To separate off the enzyme, the product was finally filtered.

### Example 4

Polyglycerol-3 caprylate

579 g of a polyglycerol characterized by the following distribution (% by weight): 0.2 of glycerol, 32.6 of diglycerol, 41.2 of triglycerol, 14.8 of tetraglycerol, 3.9 of pentaglycerol, 1.9 of hexaglycerol, 5.4 of higher polyglycerols and 363 g of caprylic acid were weighed into a three-necked flask fitted with precision glass stirrer and attached distillation bridge, and 18.8 g of Novozym® 435 were added at 60° C. The water of reaction which forms was removed in a water-jet vacuum until the acid number of the reaction mixture has dropped to a value below 2. To separate off the enzyme, the product was finally filtered.

### Example 5

Triglycerol laurate

480 g of triglycerol (obtainable from Solvay Alkali GmbH) and 401 g of lauric acid were weighed into a three-necked flask fitted with precision glass stirrer and attached distillation bridge, and 17.6 g of Novozym® 435 were added at 60° C. The water of reaction which forms was removed in a water-jet vacuum until the acid number of the reaction mixture dropped to a value below 2. To separate off the enzyme, the product was finally filtered.

### Example 6

Determination of the ratio of primary to secondary esters

The ratio of primary to secondary monoesters was determined according to the detailed method in accordance with the prior art.

The following experimental design was used as the basis:

Column: BPX 70 (from SGE); 50 m×0.22 mm internal diameter×0.25 μm film thickness

Retention gap: 5 m×0.32 mm internal diameter

Gas chromatograph: HP 6890 (FID)

Start temperature: 80° C.

Heating rate: 3° C./min

Final temperature: 260° C.

Sample preparation: Following silylation of the sample (50 mg initial weight in 1 ml of pyridine) with 0.5 ml of MSTFA over one hour at room temperature, the remainder of the MSTFA present was reacted by slowly adding 50 μl of methanol (up to 10 times).

As described, the peaks in the retention range for the diglycerol monoester were evaluated. To this end, the quotient “q” of the areal percentages of the following peaks was determined. In the case of capric acid derivatives, the areal percentages of the peaks at 39.35 and 39.45 min (linear diglycerol substituted in the 1-position) were added and divided by the areal percentages of the peak at 38.95 min (linear diglycerol substituted in the 2-position). A corresponding pattern of peaks also arose for those diglycerol monoesters which carry acid radicals other than capric radicals. In this connection, shifts in the retention times for the peaks of each individual isomer were the same, and further shifts arose depending on the measurement parameters, such as, for example, the flow rate. However, this changed neither the relative position nor the relative intensities of the peaks of the listed relevant isomers to one another. The resulting value of the quotient “q” thus always gives the molar ratio of primary to secondary esters of linear diglycerol in the products according to the invention. By way of example, the determination of this value is shown on 4 products.

For the evaluation, the area of the top peak A was used as divisor, the sum of the areas of the two back peaks B+C was used as dividend. The quotient q then directly represents the molar ratio of primary to secondary monoesters.

According to the teaching of the prior art, only the primary monoesters of diglycerol (substitution in the 1-position) and the secondary monoesters of triglycerol (selective substitution in the 2′-position) are effective in controlling Gram-positive bacteria. However, the determination of the antimicrobial activity toward microorganisms of products according to the invention shows that mixtures of primary and secondary polyglycerol esters in an effective ratio also have at least comparable, usually even significantly superior, activities. In the sense of a very efficient antimicrobial activity, preference is given here to mixtures of primary and secondary polyglycerol esters which have a “q” value between 8 and 25, particularly preferably between 10 and 20. Mixtures with “q” values outside of this range, i.e. the pure primary monoesters of diglycerol (q→∞), and the pure secondary monoesters of triglycerol (q→0) or mixtures with an ineffective ratio (q>25; q<8) have inferior activities. The effectiveness of the products according to the invention is ascertained using the challenge test (in accordance with the European Pharmaceuticals Directive).

### Example 7

Carrying out the microbiological tests:

A) Against Corynebacterium xerosis, Staphylococcus epidermidis and Candida albicans

1. Samples and material

1.1. Samples

a. Diglycerol monocaprate (D-caprate A, Solvay Alkali GmbH; comparison substance according to the prior art)

b. Diglycerol caprate (working example 1)

c. Polyglycerol-3 caprate (working example 2)

d. Diglycerol caprylate (working example 3)

e. Polyglycerol-3 caprylate (working example 4)

f. Triglycerol monolaurate (T-Laurate A, Solvay Alkali GmbH; comparison substance according to the prior art)

g. Triglycerol laurate (working example 5)

1.2. Test microbes

Corynebacterium xerosis DSM 20743

Staphylococcus epidermidis DSM 3269

Candida albicans ATCC 10231

1.3. Media used

Nutrient media:

CSL: Casein peptone-soybean meal peptone solution

CSA: Casein peptone soybean peptone-agar

Sabouraud-glucose broth/agar

Dilution liquid with inactivation additives

NaCl-peptone buffer solution with inactivator (3% of Tween® 80, 0.3% of lecithin, 0.1% of histidine, 0.5% of Na thiosulfate)

2. Method

2.1. Preparation of the test solutions

On the day before the investigation, test solutions of 0.1% (w/v) (samples a-f) and of 0.5% (w/v) (samples g, h) in CSL were prepared from each sample. For this, 100 ml of CSL were heated to 60° C. in each case in the water bath. From each sample, 0.1 g (samples a-f) and 0.5 g (samples g, h) was weighed into 100 ml of CSL in each case at 60° C. The preparations were shaken vigorously by hand and left overnight at 30° C. in an incubator.

2.2. Preparation of the test microbe suspensions

Cultivate Corynebacterium xerosis over 3 to 4 days. Isolate other microbes in broth or by elutriation.

2.3. Contamination of the samples and determination of the reduction in number of microbes

For each test microbe, 20 ml of each test solution were introduced into sterile 50 ml brown glass bottles with glass beads and contaminated with 0.2 ml of microbe suspension. As controls, 20 ml of CSL were carried over per test microbe without sample. The contaminated samples were shaken for 3 min on a shaking machine and kept in an incubator at 30° C. until removed.

At the removal points (1, 2, 3, 24 and 48 hours), 1 ml was taken from each preparation and transferred to in each case 9 ml of NaCl-peptone buffer solution with inactivator and the colony number was determined.

The 0 hours values given were the colony numbers of the test microbe suspension used taking into consideration the 10⁻² dilution upon sample contamination.

3. Results

The individual results of the samples are shown in the diagrams below. Also shown on each diagram are the microbe populations of an active-ingredient-free blind sample as control value after incubation for 24 hours.

B) Against Malassezia furfur

In the same procedure as described under A, the effectiveness of diglycerol caprylate, as prepared in working example 3, is tested against M. furfur. M. furfur is causally related to the formation of dandruff.

Diglycerol caprylate was dissolved in water to give a solution containing 3.0% by weight. This solution was treated with microbial suspension, homogenized by shaking and incubated at 30° C. A second solution without the addition of diglycerol caprylate was also prepared as control.

The following results were obtained:

| | | | | | |
|---|---|---|---|---|---|
| Sampling, time (h) | 0 | 1 | 2 | 4 | 24 |
| Control, no. of microbes/ml | 1 × 10⁵ | n.d. | n.d. | n.d. | 1 × 10⁴ |
| 0.3% Diglycerol caprylate, no. | 1 × 10⁵ | <10 | <10 | <10 | <10 |
| of germs/ml | | | | | |

n.d. = not determined

### Example 8

Use in formulations

Formulations in which the products according to the invention can be used are given below.

Formulation 1: Clear Deodorant Pumpspray

| | |
|---|---|
| Phase A: | |
| Product from example 4 | 0.30% |
| Trideceth-12 | 2.00% |
| Dipropylene glycol | 4.00% |
| Perfume | 0.90% |
| Phase B: | |
| Water | ad 100.00 |
| Preservative | q.s. |
| Citric acid (50% strength) | q.s. |

The constituents given under phase A are combined with stirring in the order given and then slowly topped up with water (phase B). The pH is adjusted to 5.5 with citric acid.

Formulation 2: O/W emulsion (sprayable)

| | |
|---|---|
| Phase A: | |
| Glycerol stearate (and) Ceteth-20 | 3.00% |
| (e.g. TEGINACID ® H, Degussa) | |
| Stearyl alcohol | 1.00% |
| Product from example 4 | 0.30% |
| Dimethicones | 0.50% |
| Cetearylethyl hexanoate | 4.00% |
| Caprylic/capric triglyceride | 4.00% |
| Phase B: | |
| Glycerol | 3.00% |
| Water | ad 100.00% |
| Citric acid (50% strength) | pH = 6-7 |
| Preservative | q.s. |
| Perfume | q.s. |

Phases A and B are heated to 70 to 75° C. Phase A is added with stirring to phase B and then homogenized. The mixture is cooled with stirring to 30° C.

Important: If phase A is to be introduced initially, phase B must be added without stirring.

Formulation 3: Clear Deodorant Roll On

| | |
|---|---|
| Phase A: | |
| Product from example 4 | 0.30% |
| Trideceth-12 | 2.00% |
| Dipropylene glycol | 2.00% |
| Perfume | 0.50% |
| PEG-14 dimethicones | 1.00% |
| Water | ad 65.00% |
| Phase B: | |
| Hydroxyethylcellulose (2% in water) | 35.00% |
| Preservative | q.s. |
| Citric acid (50% strength) | q.s. |

The constituents given under phase A are combined with stirring in the order given. Phase A is added with stirring to phase B. The pH is adjusted to 5.5 with citric acid.

Formulation 4: Anionic household cleaner (concentrate)

| | |
|---|---|
| Phase A: | |
| Product according to the invention | 4.00% |
| Ethanol | 10.00% |
| Trideceth-12 | 5.00% |
| Cocamidopropylbetaine (˜38% active | 13.20% |
| ingredient content) | |
| Sodium lauryl ether sulfate | 35.80% |
| Phase B: | |
| Water | ad 100.00% |

The constituents given under phase A were combined with stirring in the order given and then slowly topped up with water (phase B).

### Example 9

Cosmetic application test

Two formulations were used. These are formulation 2 from example 6 and, as placebo, the same formulation in which the product according to the invention (from example 4) was been replaced by nonesterified polyglycerol with the same distribution. The armpit odor of 20 subjects was tested before and after application of formulation 2 or the placebo formulation by three experts. In detail, the test involves the following steps:

1. The armpit is washed with soap, the odor is evaluated by experts.

2. The product applied used once in one armpit. After 6 and 24 h, the odor is tested and the difference is evaluated.

The result of this investigation is that, both after 6 and also after 24 hours' use, a significant improvement in the odor of the armpit treated according to the invention compared with the placebo-treated armpit is established.

### Example 10

Preserving a food

Potato salad consisting of 750 g of cooked and finely chopped potatoes, 25 g of finely chopped onions, 1.2 g of cooking salt, 10 ml of vinegar (comprising 6% acetic acid) and 200 g of mayonnaise was treated with 0.5% of the polyglycerol ester from example 4. To check on bacteria and yeasts the potato salad was stored for 72 hours at 20° C. Afterwards the following numbers of germs were determined:

Potato salad without polyglycerol ester: 1.2·10⁶ number of germs/ml

Potato salad with polyglycerol ester: 1.3·10³ number of germs/ml

To check on yeasts and fungi the potato salad was stored for 72 hours at 25° C. Afterwards the following numbers of germs were determined:

Potato salad without polyglycerol ester: 6.7·10⁴ number of germs/ml

Potato salad with polyglycerol ester: 2.5·10¹ number of germs/ml

The potato salad without polyglycerol ester showed after 96 hours storage clearly visible blueish mould, whereas the potato salad with polyglycerol ester was visually unchanged.

The above description of the invention is intended to be illustrative and not limiting. Various changes or modifications in the embodiments described herein may occur to those skilled in the art. These changes can be made without departing from the scope or specification of the invention.

1. A composition for controlling microorganisms, which comprises an effective amount of a mixture comprising primary and secondary fatty acid esters of polyglycerol wherein the molar ratio of primary esters of polyglycerol to secondary esters of polyglycerol from about 8:1 to about 25:1.

2. The composition as claimed in claim 1, wherein the ratio is from about 10:1 to about 20:1.

3. The composition as claimed in claim 1, wherein the polyglycerol is obtained by condensation of glycerol, by hydrolysis and condensation of epichlorohydrin or by polymerization of glycidol.

4. The compositions as claimed in claim 1, wherein the polyglycerol is of the formula
HO—CH₂—CH(OH)—CH₂—O—[CH₂—CH(OH)—CH₂—O]ₙ—H
wherein n is 1 to 9.

5. The composition as claimed in claim 1, wherein the fatty acid ester has at least 6 carbons and is straight-chain or branched and, optionally, contains OH groups and/or double bonds.

6. The composition as claimed in claim 1, wherein the ratio is from 8:1 to 25:1.

7. A composition for controlling microorganisms as claimed in claim 1, wherein the fatty acid portion in the fatty acid esters are residues of caproic acid, caprylic acid, capric acid, 2-ethylhexanoic acid, undecylenic acid, lauric acid myristic acid or a mixture of any of the foregoing.

8. A method for controlling the growth of Gram-positive bacteria, Gram-negative bacteria, mycobacteria, dermatophytes, yeasts, fungi and hyphal fungi, viruses and spores or disinfecting an area where said Gram-positive bacteria, Gram-negative bacteria, mycobacteria, dermatophytes, yeasts, fungi and hyphal fungi, viruses and spores reside which comprises applying or adding to said area a composition according to claim 1.

9. A disinfectant, disinfectant cleaner, sterilizing composition, antiseptic or preservative which comprises an effective amount of a composition according to claim 1.

10. A method for preserving foods which comprises an effective amount of a composition according to claim 1 to said foods.

11. A food packaging material which comprises a composition according to claim 1.

12. A cosmetic formulation which comprises a composition according to claim 1.

13. A cosmetic formulation according to claim 12 which is for body cleansing, or body care.

14. The cosmetic formulation according to claim 12, which is for treating acne or blemished skin.

15. A deodorant which comprises an effective amount of a composition according to claim 1.

16. A method for reducing body odor in a subject which comprises applying a deodorant according to claim 15 to said subject.

17. A haircare product which comprises an effective amount of a composition according to claim 1.

18. A method for reducing the formation of dandruff in a subject in need thereof which comprises applying a hair care product according to claim 18 to said subject.

19. A method for treating blemished skin or acne in a subject in need thereof which comprises applying to the skin of the subject an effective amount of a formulation according to claim 14.
